(19) United States
(12) Patent Application Publication
(10) Pub. No.: US 2012/0220819 A1
(43) Pub. Date: Aug. 30, 2012

(54) Stroller Mat

(76) Inventors: Gerald Michael Madl, West Sacramento, CA (US); Shani Madl, West Sacramento, CA (US)

(21) Appl. No.: 13/035,910

(22) Filed: Feb. 25, 2011

(51) Int. Cl. A61M 21/02

(52) U.S. Cl. 600/26

(57) ABSTRACT

The invention provides a mat providing a surface for creating movement within a stroller having wheels rolled over the mat, where the mat has an upper and lower surface, the upper surface comprising a texture surface and spaced vertical extensions elevated over the textured surface, and where the mat is produced of a non-shock absorbing material.

### BACKGROUND OF THE INVENTION

1. Field of the Invention

The present invention relates generally to floor mats, and in particular to a mat that creates random movement and varied intensity when used in combination with an infant stroller.

2. Description of Related Art

Many newborns require assistance in falling asleep. While colicky and fussy babies have particular issues in regard to calming down, most babies sooner or later experience times when they have a hard time falling asleep. It has been proposed that there is a “calming reflex” that can be triggered by certain and specific vibrations or movements.

In any case, parents have instinctively employed various tools or “tricks” to achieve the sometimes elusive goal of calming their babies, with varying degrees of success. Holding a baby all the time can become very tiring, or in case of multiples may even be impossible. Also, because of fear of “Shaken Baby Syndrome” many parents are afraid of the vigorous movement that is required to trigger the calming reflex.

Various devices have been employed to also accomplish this goal, also with varying degrees of success. These have ranged from “traditional” aids such as clothes dryers and late night car rides, or just simply holding the baby and rocking/bouncing them, to elaborate manufactured devices, such as vibrating apparatuses, bouncers or swings. Many of these methods or devices are not very practical, ineffective or not necessarily safe. Baby swings and bouncers oftentimes do not work and are also too inconvenient to bring along. Driving around in a vehicle while being tired is not a good option.

Taking an infant for a stroll is known to be particularly soothing, but often times it is not practical to do so (e.g. bad weather, late night or lack of safe walking area). Some babies fall asleep while being carried around but they wake up as soon as one sits down to take a break. For these reasons, taking an infant for a regular walk may not work as a predictable and reliable way to put a baby to sleep.

Is has also been observed that many babies like the uneven and random surfaces that can be encountered during such walks. However, not all surfaces are created equal. Sometimes the ride is just too smooth and other times it will not create enough of a “bounce”. No studies have been designed to determining the perfect surface for lulling a baby to sleep in a stroller walk. However, if such a surface or floor could be provided within the safety and comfort of one's home, a baby could be soothed at any time and parents will not be dependent on their location or inclement weather.

The idea of providing a surface for this purpose is not new, for instance, McNab, Patent Application Publication No. 2005/0282465, suggests a molded mat for cooperation with a baby stroller. However, its design is focused more on the appearance of the map than on any principles aimed for optimizing the stroller “bounce”. McNab does not provide the necessary traits to work for most strollers, and by utilizing the mat for purposes other than child pacification, it loses its effectiveness in for that specific purpose.

### SUMMARY OF THE INVENTION

These and other features and advantages of this invention are described in, or are apparent from, the following detailed description of various exemplary embodiments of the apparatus and methods according to this invention.

A system and method are disclosed for a mat providing a surface for creating movement within a stroller having wheels rolled over the mat, where the mat has an upper and lower surface, the upper surface comprising a texture surface and spaced vertical extensions elevated over the textured surface, and where the mat is produced of a non-shock absorbing material.

In a further embodiment, the mat is produced of a unitary molded polymer.

In a further such embodiment, the non-shock absorbing material is semi-rigid plastic.

In a further such embodiment, the non-shock absorbing material is a plastic material selected from the group of plastics consisting of acrylic, polycarbonate, polypropylene, and polyvinylchloride.

In a further such embodiment, the non-shock absorbing material is a plastic having a Rockwell hardness of between about 75 and about 95.

In a further aspect, the non-shock absorbing material is a rigid plastic.

In one such version, the mat has a hinged region.

In a further such version the mat may be produced of HDPE plastic.

In a further embodiment, the textured surface has texture formed of waves.

In a further such embodiment, the distance between wave peaks is about 4.4 mm.

In a further such embodiment, the wave height is about 2 mm.

In a further such embodiment, the textured surface has texture formed of nibs.

In a further such embodiment, the textured surface has texture formed of small bumps.

In a further such embodiment, the textured surface has texture formed of peaks and valleys.

In a different embodiment, the mat has external dimensions of at least about 75 cm by 150 cm.

In a further such embodiment, the mat has external dimensions of at least about 100 cm by 175 cm.

In a one embodiment, the mat has a thickness of at least about 3 cm.

In a further such embodiment, the mat has a thickness of at least about 4 cm.

In a still further such embodiment, the mat has a thickness of at least about 5 cm.

In a different embodiment, the bottom surface is flat.

In a still different embodiment, the bottom surface is textured.

In one embodiment, the vertical extensions are spaced in parallel rows.

In a further such embodiment, the vertical extensions are spaced unevenly across the upper surface.

In a different embodiment, the vertical extensions are circular bumps.

In a still different embodiment, the vertical extensions are pyramids.

In another different embodiment, the vertical extensions further comprise a surface groove, or plurality of grooves.

In a further embodiment, each surface groove has a width of about 3 mm.

In a further such embodiment, the surface grooves are spaced with about 2 mm between edges.

In a still further embodiment, the vertical extensions have a height of about 4 mm.

In still further embodiment, the vertical extensions have a height of about 6 mM.

In a further such embodiment, the vertical extensions have a height of about 8 mm.

In another embodiment, the distance across the base of the vertical extension is about 24 mm.

In a different embodiment, the distance between vertical extensions is about 10 mm.

In a further such embodiment, the distance between vertical extensions is about 20 mm.

In a still a further such embodiment, the distance between vertical extensions is about 30 mm.

The invention also discloses a method for creating a pacifying action in a stroller comprising the steps of moving a stroller over such a mat, such that the movement of a stroller wheel across the textured surface and the vertical extensions provides a combination of a constant slight vibration and less predicable and intermittent jolts to the stroller.

The simple yet very practical design of the above described mat can be utilized anywhere and anytime a parent needs help to get their baby to calm down and fall asleep. It is also mobile and can easily be taken to any location or stored when not in use.

These and other features and advantages of this invention are described in, or are apparent from, the following detailed description of various exemplary embodiments of the apparatus and methods according to this invention.

### BRIEF DESCRIPTION OF THE DRAWINGS

A more complete understanding of the present invention and the attendant features and advantages thereof may be had by reference to the following detailed description when considered in conjunction with the accompanying drawings wherein:

FIG. 1 is a plan view of the top of the surface of the mat.

FIG. 2 is a partial enlarged top view of a corner of the mat in FIG. 1, illustrating the grooved vertical extension and the wave pattern of the mat surface.

FIG. 3 is a side view of the mat.

FIG. 4 is a partial enlarged side view of the mat illustrating the wave pattern and the grooved bumps of the mat.

### DETAILED DESCRIPTION OF THE INVENTION

There are a multitude of mats or tiles, and produced from a great variety of materials, but there is none that provides all the necessary attributes to pacify infants like the design of this invention.

Mats merely providing an uneven surface may be used to simulate some aspects of a simple walk outside. The present mat, however, is specifically designed to improve on, and be more efficient than, a walk outside, and is especially appropriate for babies who are particularly fussy or colicky. Not every surface will work for infants with these particular traits. A mat for child pacification under such conditions has to have specific attributes.

Various mats/tiles available for purposes other than specifically for child pacification were evaluated, but none exhibited the necessary traits. Such products include office chair mats, anti-fatigue mats, dust control mats, pedestrian safety tiles, tactile tiles, non-skid mats, rubber floor mats, acoustic floor mats, etc. Many different textures and materials were tested, before the unique surface pattern was adopted, as described below.

Careful analytical observation and an understanding of the movement required lead to the specific design disclosed herein. A combination of specific bumps in combination with a wave pattern over the even surface of the mat is necessary to make a stroller bounce three-dimensionally and also vibrate when being rolled on the mat. Because the path of a stroller over the mat is never the same, the random bounce it creates makes it feel natural for the baby. In the case of a mat, the intensity will depend on how fast the rolling movement is replicated, in a back and forth fashion. Thus, while a very slight vibration is created by the wheels over the even portion of the map having a wave pattern, the intermittent bumps are required to duplicate the effect of the natural “bounce” produced by a stroller on a walk outdoors. Both of these movements, vibration and intermittent bumps, are necessary for the mat to work as intended in optimizing baby pacification.

The height and spacing of the ridges and bumps are also important, in order to create a movement that allows the user to utilize most strollers available today. The material of the mat is preferably a semi-rigid plastic or similar material necessary to create a hardness that is necessary for vibration/bounce intensity. Stroller wheels materials can range anywhere from hard plastic to air-inflated tires. To accommodate the wide variety of these wheels and still achieve the vibration/bounce the material of the mat has to be non-shock absorbing.

The mat also needs to have an external dimension that is small enough to allow it to be folded or rolled for practicality reasons, such as traveling, shipping and storing. In order to accommodate most strollers, the preferred dimensions of the mat for this purpose are on the order of at least about 75 cm×150 cm (30″×60″).

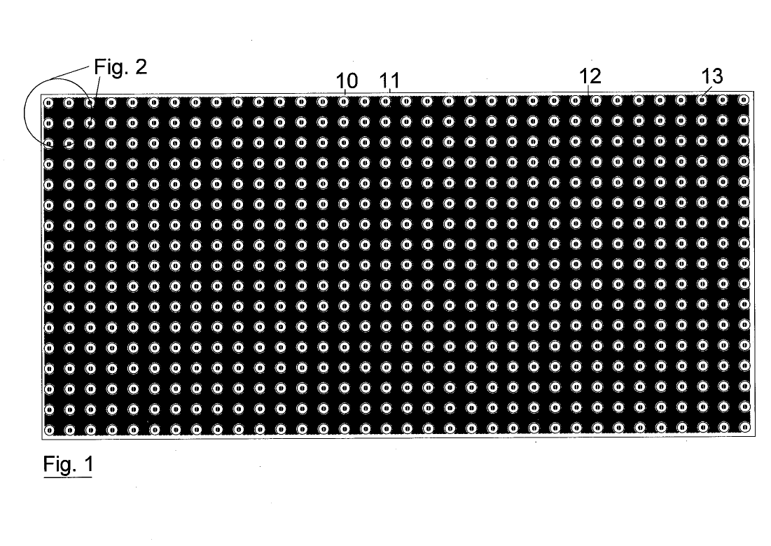

Having explained the principles of the mat, attention is now drawn to FIG. 1, which shows an exemplary floor mat (10) of the invention produced of a semi-hard substrate (11). The harder substrate is important, so that the mat material itself absorb as little vibration and shock as possible during wheel rotation. The dimensions of the mat (10) are approximately 75 cm wide by 150 cm long but could vary by +/−25 cm in order to accommodate various sized strollers that are rolled back and forth over it perpendicular to the wave pattern (12). The general thickness (14) of the mat (10) is dependent on the hardness of the material used but can be on the order of about 4 mm, +/−1 mm. The bottom of the mat (10) may be flat and without any specific texture other then created during the manufacturing process, or may have skid preventing features or texture applied.

The wave pattern (12) design is continuous throughout the whole mat (10) over the even surface or substrate (11) except of the locations of the upwardly protruding bumps (13) and the perimeter margin of the mat (10). The wave pattern (12) runs parallel to the short side of the mat (10) and therefore allows the wheels of a stroller to roll perpendicular over it, creating a slight vibration.

A plurality of upwardly protruding vertical extensions, or bumps (13), is also part of the surface of the mat (10). The bumps (13) are spaced in parallel rows equidistant to each other. The tops of the bumps may further be provided with grooves (17) to further add vibrations as the stroller wheels travel at a path over the top of the bumps (13).

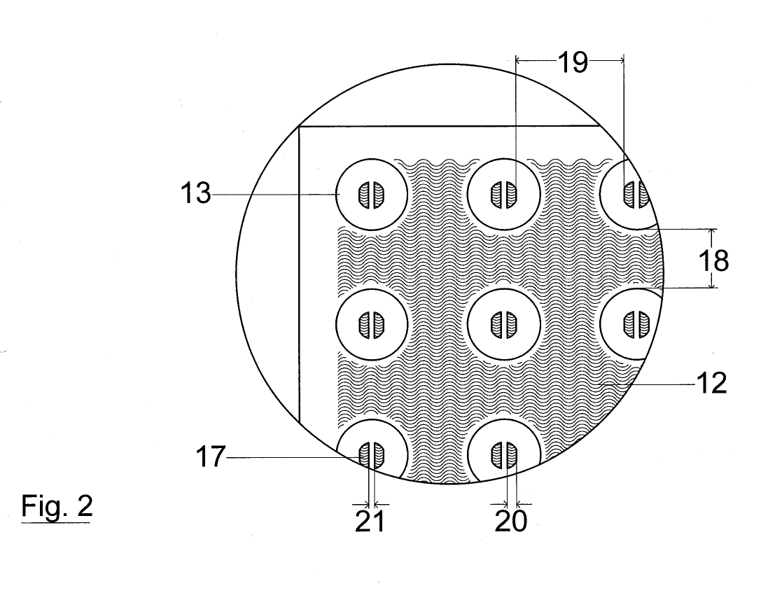

FIG. 2 provides a magnified view of the corner of the mat of FIG. 1, better illustrating the wave and bump pattern produced on the surface. The size of the optimal grooves (17) formed on top of the bumps (13) can vary somewhat, depending on the degree of bounce versus vibration desired. The width (20) of each groove is 3 mm (+/−1 mm) with a spacing of 2 mm (+/−1 mm) between them (21).

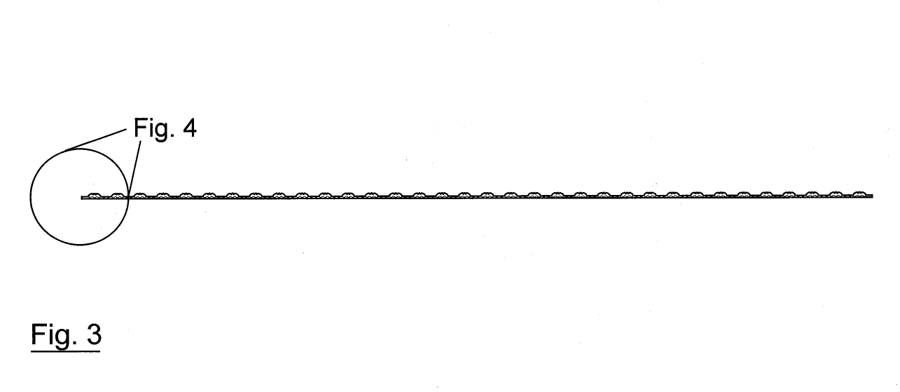

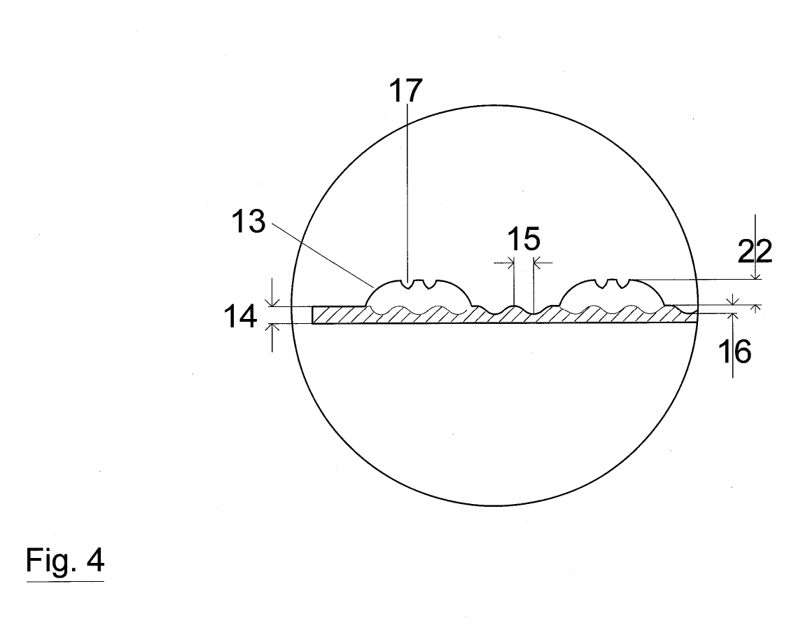

FIGS. 3 and 4 provide a side view of the map, showing the wave and bump pattern in cross section.

As illustrated in the magnified view provided by FIG. 4, in order to accommodate various stroller wheel sizes the horizontal distance (15) from the highest point of the wave pattern (12) to the lowest point is 4.4 mm but can vary by +/−2.2 mm. The vertical distance (16) from the highest point of the wave pattern (12) to the lowest point is preferably about 2 mm, though this may vary by +/−1 mm.

The total height (22) of the dome (13) from the top of the wave pattern to the dome's highest point is preferably about 6 mm, but can vary by +/−2 mm. The diameter of the dome at its base is preferably about 24 mm but can vary by +/−3 mm.

The dome shape could be substituted with various other shapes such as pyramids, cones, etc. Scaling such similar shapes accordingly could create similar jolts, or bumps, and should be considered within the scope of this invention.

The wave pattern (12) could be substituted with various other subtle surface patterns, such as smaller bumps, nibs, groves, or peaks and valleys, etc. Spacing such similar patterns accordingly could create similar vibrations and should be considered within the scope of this invention.

While wheel movement over the wave patterns (12) of the mat (10) as well as the grooved tops (17) of the bumps (13) provide a constant slight vibration, the bumps (13) create the less predicable and intermittent jolts, bounces and bumps to the baby, via the stroller wheels conveying the action throughout the stroller frame.

As noted in FIG. 4, the steepness to the wall of the bumps (13) can be varied, as this leads to a greater or lesser resistance of the stroller wheels to travel over the top of the bumps. Such resistance aids in creating a horizontal component in the movement, and can be increased with the severity of the steepness of the bumps.

In reference again to FIG. 2, the spacing (18) of the base of the bumps (13), can vary, but is preferably on the order of about 20 mm, +/−10 mm. While the widthwise (18) spacing of the bumps (13) allows for various widths of stroller wheels, the lengthwise spacing of also 20 mm (+/−10 mm) is necessary for a high frequency in jolts, or bumps, without creating too great of a pause when rolling a stroller slowly back and forth over the mat (10).

As seen in reference to FIGS. 2 and 4, the distance (19) from the edge of the serrated groove of one dome to the edge of the serrated groove of the next dome is about 36 mm (+/−4 mm), and is important to accommodate various diameters of stroller wheel sizes and directly relates to the vertical component of the stroller movement.

The described mat surface provides the occupant of a stroller with both, a steady vibration, and random three-dimensional jolts, or bumps.

The mat is preferably produced of a semi-rigid plastic, such as acrylic, polycarbonate, polypropylene, or polyvinylchloride (with a Rockwell hardness of between about 75 and 95) to maximize the delivery of vibrations and jolts created when a stroller is rolled over its surface. This is necessary because there is a certain intensity required to trigger a natural calming reflex. The device could also be produced from hard plastic such as HDPE with a hinge mechanism. Smaller tile type pieces could be folded with a hinge mechanism while still providing the same shipping and transportability advantages. Stylish designs as well as using materials that have no smell make this product usable inside the home or outside. Some products, like rubber, can have a strong smell and are not a good choice for inside the house.

In any case, the semi-rigid mat has to be hard enough to maintain its shape when placed on a carpet or any other soft surface. In a preferred aspect, the mat is also flexible enough to be rolled for storage and shipping purpose.

High frequency vibrations are necessary to create a soothing environment for the infant in the stroller. This is achieved by utilizing a wave pattern of certain peak to valley distance as well as their dimensions. The wave pattern runs perpendicular to the direction of the stroller being pushed from front to back. The wave pattern is preferred but vibrations could also be produced by perpendicular running grooves in a mat.

Intense jolts are necessary in addition and in combination of the vibrations to trigger the calming reflex which are created by the dome shaped protrusions. The bumps have grooves to achieve the additional and necessary vibration when the wheels of the stroller travel over the apex of the bumps. Height, spacing and shape are pertinent to produce such movement.

The shapes of the bumps as well as their dimensions are found to be the best possible solution to create the desired chaotic and random three-dimensional movement. The spacing of said bumps is necessary to maximize the vertical component of the movement.

Randomness of movement is achieved with this grooved dome/wave pattern design because it is pertinent to make the movement feel natural for the infant.

The intensity of the movement can easily be controlled by how fast or slow one pushes the stroller back and forth over the mat while standing or being seated.

There is no power required to use this mat. It can be taken anywhere the user wishes.

With the described mat, there is no risk of Shaken Baby Syndrome. Parents using this mat do not have to worry about shaking their babies too intensely. The most intense movement over the mat would still be safe for any infant.

While the present invention is described with reference to the illustrations for specific applications, it should be understood that the invention is not limited to those applications. Those skilled in the art with access to invention described herein will recognize additional modifications, applications, and embodiments within the scope thereof and additional fields in which the invention would be of significant utility.

What is claimed is:

1. A floor mat having a surface for creating movement within a stroller having wheels rolled over the mat,
wherein the mat has an upper and lower surface, the upper surface comprising a texture surface and spaced vertical extensions elevated over said textured surface, and
wherein the mat is produced of a non-shock absorbing material.

2. The mat of claim 1, wherein the non-shock absorbing material is semi-rigid plastic.

3. The mat of claim 1, wherein the non-shock absorbing material is a plastic having a Rockwell hardness of between about 75 and about 95.

4. The mat of claim 1, wherein the non-shock absorbing material is a rigid plastic.

5. The mat of claim 4, wherein the rigid plastic is HDPE.

6. The mat of claim 1, wherein the textured surface has texture formed of waves.

7. The mat of claim 1, wherein the distance between wave peaks is about 4.4 mm.

8. The mat of claim 1, wherein the wave height is about 2 mm.

9. The mat of claim 1, wherein the mat has external dimensions of at least about 75 cm by 150 cm.

10. The mat of claim 1, wherein the mat has a thickness of at least about 3 cm.

11. The mat of claim 1, wherein the bottom surface is flat.

12. The mat of claim 1, wherein the bottom surface is textured.

13. The mat of claim 1, wherein the vertical extensions further comprise a surface groove.

14. The mat of claim 1, wherein the surface groove has a width of about 3 mm.

15. The mat of claim 1, comprising two surface grooves.

16. The mat of claim 1, wherein the surface grooves are spaced with about 2 mm between edges.

17. The mat of claim 1, wherein the vertical extensions have a height of about 4 mm.

18. The mat of claim 1, wherein the distance across the base of the vertical extension is about 24 mm.

19. The mat of claim 1, wherein the distance between vertical extensions is about 10 mm.

20. A method for creating a pacifying action in a stroller comprising the steps of moving a stroller over the mat of claim 1, wherein the movement of a stroller wheel across the textured surface and the vertical extensions provides a combination of a constant slight vibration and less predicable and intermittent jolts to the stroller.
